(19) United States
(12) Patent Application Publication
(10) Pub. No.: US 2007/0243567 A1
(43) Pub. Date: Oct. 18, 2007

(54) Beta cell mimicking control algorithm for artificial pancreas

(76) Inventor: Syhhong Chang, Fullerton, CA (US)

Correspondence Address: LAW OFFICES OF CLEMENT CHENG, 17220 NEWHOPE STREET #127, FOUNTAIN VALLEY, CA, 92708, US

(21) Appl. No.: 11/786,596

(22) Filed: Apr. 12, 2007

Related U.S. Application Data
(60) Provisional application No. 60/791,695, filed on Apr. 13, 2006.

(51) Int. Cl. A61K 38/28; C12Q 1/54; G06F 19/00

(52) U.S. Cl. 435/14; 702/19; 514/3; 600/300

(57) ABSTRACT

The invention is a method of controlling an artificial pancreas. The method includes reading and recording a current plasma glucose level. The method includes computing glucose in beta cell if a current plasma glucose level is greater than a threshold value, and continuing the step of reading and recording a current plasma glucose level if a current plasma glucose level is less than a threshold value. The method includes calling ATP functions for interpolation. The method includes calculating a holding capacity. The method includes calculating a synthesized insulin amount. The method includes comparing amount of insulin with the holding capacity. The method includes releasing insulin if the amount of insulin is greater than the holding capacity, and not releasing insulin if the amount of insulin is less than the holding capacity.

This application claims provisional priority 60/791,695 filed Apr. 13, 2006 for same inventor Mr. S. Chang entitled The Cell-Mimicking Control Algorithm for Artificial Pancreas and Simulation of a Complete Closed-Loop Glucose-Insulin Kinetics, which is incorporated herein by reference.

### BACKGROUND OF THE INVENTION

Diabetes is an illness where inadequate insulin secretion causes high blood sugar. Insulin is one of the important human hormones (messengers). It informs your body to perform glucose metabolism after food intake. Without insulin, glucose stays in body's extracellular fluids (plasma and interstitial fluid) and causes hyperglycemia (high blood sugar). Too much blood sugar can damage blood vessels, lead to heart disease, blindness, kidney failure and amputations.

Typically, the human body produces insulin in the beta cell of the pancreas. When beta cells loses their function to produce insulin due to a gene defect it is called Type I diabetes or Insulin Dependent Diabetes Mellitus (IDDM). This happens when a patient is very young and usually it starts when patient is around 12 years old. There are two million patients who are mostly young adults in the United States alone. They have a daily reliance upon insulin shots or an insulin pump.

Patients of Type I diabetes usually carry a glucose monitor and insulin shots or an insulin pump. Before any meal, they have to prick one of their fingers or other parts of the body to have a glucose reading. They also have to estimate the amount of food they are going to eat. Then, they determine how much insulin to pump into their body. For those patients who carry a shot with preset dose, they have to be on a strict diet schedule to avoid hyperglycemia. Meal size, types of foods, body absorption, activity after meal as well as a person's mood all affect patient's glucose metabolism. It really is extremely difficult for those poor kids to have their blood sugar level under control.

Therefore, patients of Type I diabetes have frequent insulin overdose. This leads to hypoglycemia (blood sugar is too low). Without immediate and proper care, a patient can have a coma and severe brain damage. It will be much easier and healthier for Type I patients to skip all the guessing, do whatever activity they want and have a more flexible and relaxed daily schedule.

With today advanced technology, both glucose monitoring and insulin dispensing have become more accurate, less invasive and therefore can be done more frequently. Today, the Continuous Glucose Monitor (CGM) provides real time glucose monitoring with a time interval of minutes. Beta cells in the human body releases insulin by sensing glucose level continuously. More sophisticated insulin dispensing devices, such as the one using ultrasound wave to pump insulin into body, offers minute amounts and still precise amounts of insulin to be released each second. A normal beta cell functions continuously and nonstop to provide adequate insulin release.

An Integrated Artificially Pancreas System (IAPS) is an integrated system which provides continuous glucose monitoring and dispenses insulin in a way a normal pancreatic beta cell does. It consists of three components: a Continuous Glucose Sensor (CGS), an Insulin Dispensing Device (IDD) and a Control Unit (CU) where control algorithm is attached along with a display panel and data storage etc. FIG. 1 shows a setup of a standard artificial pancreas on a human body. CGS can be either external or implantable. Ideally, patients using IAPS need not to worry too much about what and how they eat, what kind of activity they do or even with no activity at all hours after a meal. A well designed control algorithm should make an IAPS perform exactly in that way.

This invention focuses on the control algorithm of an artificial pancreas. The integrated system determines the amount of insulin release upon reading the level of plasma glucose at that same moment with real time detection and action. An algorithm is a set of logically controlled math formulas which enables quantitative actions. Control algorithm of artificial pancreas provides a linkage between the blood-glucose monitoring device and insulin-dispensing device.

Most current available algorithms base on the variation of plasma glucose level to determine the amount of insulin release. Many derivative and/or integral methods perform in that way. However, pancreatic beta cells detect interstitial fluid glucose for its insulin release. Another type of algorithm assumes plasma glucose decreases exponentially from a maximum value and estimate a proper insulin release amount, namely bolus. It has to provide a basal insulin release amount too. This kind of algorithm fails to handle the cases where plasma glucose increases due to body's reabsorption of glucose or due to additional food intake after a meal. While some engineers use a concept of neural networks to randomly search the behavior of each beta cell, there are other engineers that try a Kalman Filter because they treat small fluctuations of glucose variation as measurement errors. However, all the above approaches lack considerations on the dozen bioprocesses involved in glucose metabolism of human body.

### BRIEF DESCRIPTION OF THE DRAWINGS

FIG. 1 shows the three components of an Integrated Artificial Pancreas system (IAPS).

FIG. 2 summarizes the 12 bioprocesses within human body which are involved in glucose-insulin kinetics.

FIG. 3 shows effect of diffusion on glucose and insulin.

FIG. 4 shows the changes of GLUT2, GK, ATP and the outline of Gated Reservoir as function of glucose level of pancreatic fluids.

FIG. 5 shows flow chart of the beta cell mimicking control algorithm based on GRM.

FIG. 6 shows plasma glucose and plasma insulin variations for a resting case versus an exercise case.

FIG. 7 shows the case of patient with Insulin Resistance (IR).

### DETAILED DESCRIPTION OF THE INVENTION

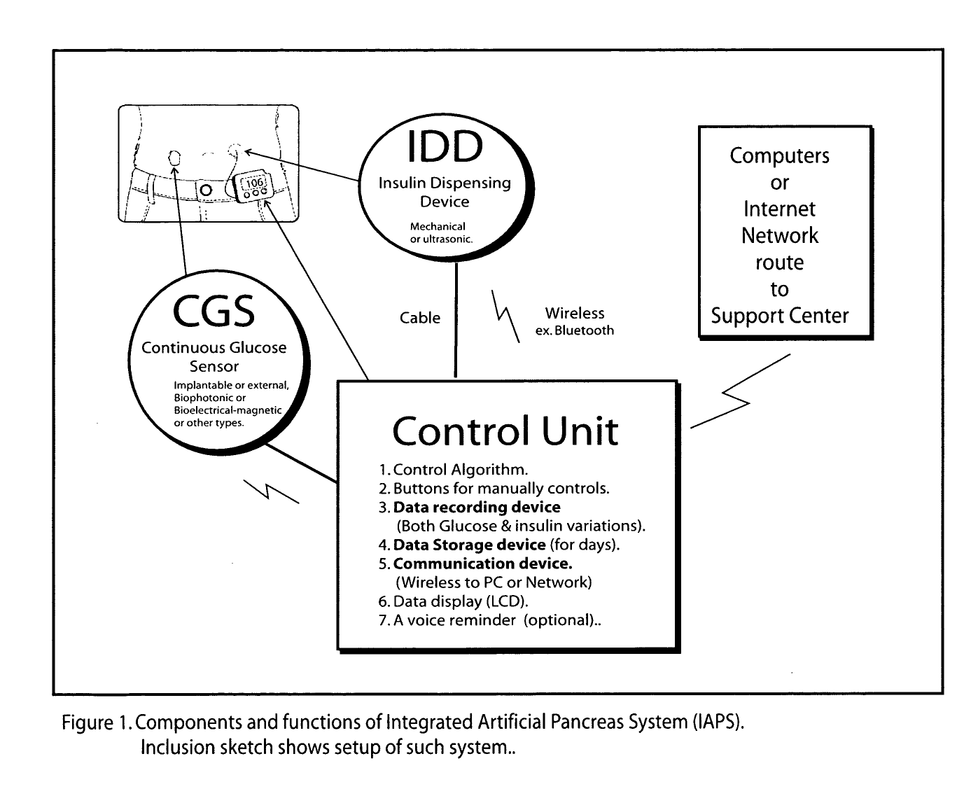

FIG. 1 shows the three components of an Integrated Artificial Pancreas system (IAPS). 1. Continuous Glucose Sensor (CGS), 2. Insulin Dispensing Device (IDD). 3.Control Unit (CU). CGS detect plasma glucose level for every time interval and send signal to CU. CU record this signal and input to the control algorithm attached into it. An insulin release amount can be then computed to instruct IDD to act for that time interval. CU can have a display panel to show glucose level, IAPS status, warnings to patient etc. CGS can be external or implantable. Sending signal between CGS and CU or between IDD and CU can use either wire or wireless. Beside receiving signal from CGS and sending signal to IDD, CU should includes also a few other devices. A. a recording device for data storage (both glucose variation and insulin released amount). B. a display panel for patient to view the status of his glucose metabolism. C. a few buttons for patient to manually control the unit. D. a communication device to communicate with other systems and/or networks for algorithm update, parameters adjustment and data relay for further analysis.

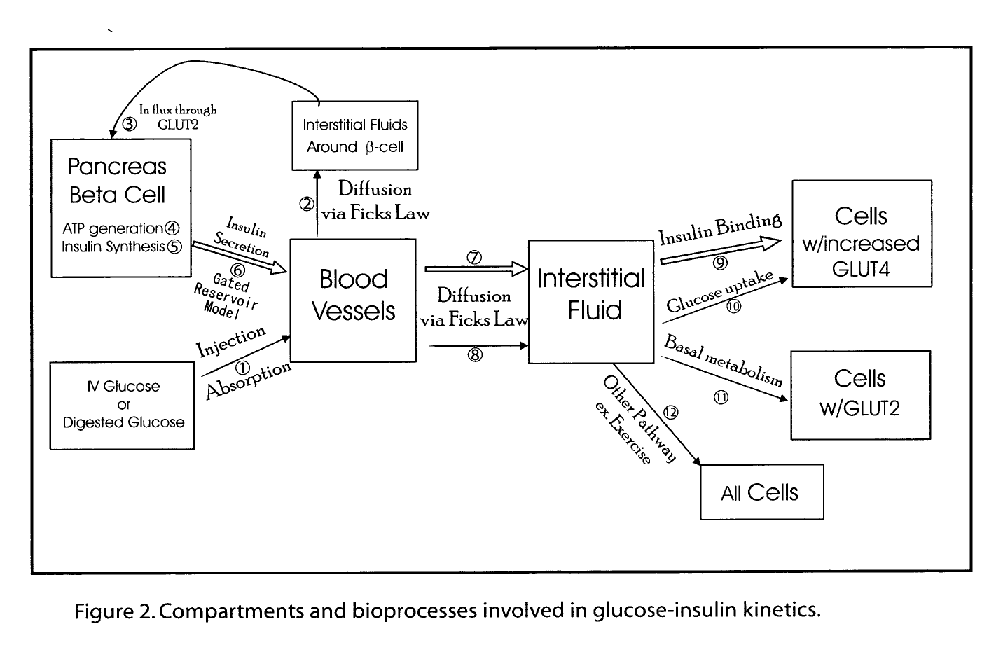

FIG. 2 summarizes the 12 bioprocesses within human body which are involved in glucose-insulin kinetics. There are essentially five compartments: Blood vessels, beta cells interstitial fluids, beta cells, target cells and target cells interstitial fluids. Glucose first enters blood vessels through intestinal absorption. It is delivered and diffused to interstitial fluids surrounding cells of entire body. It then enter beta cells and target cells. Beta cells respond to glucose changes in surrounding fluids and release insulin accordingly. Target cells, received the message through insulin binding to its membrane receptors, starts to uptake glucose. Lower glucose level changes the response of beta cells and processes continue until glucose level is reached around 90 mg/dL.

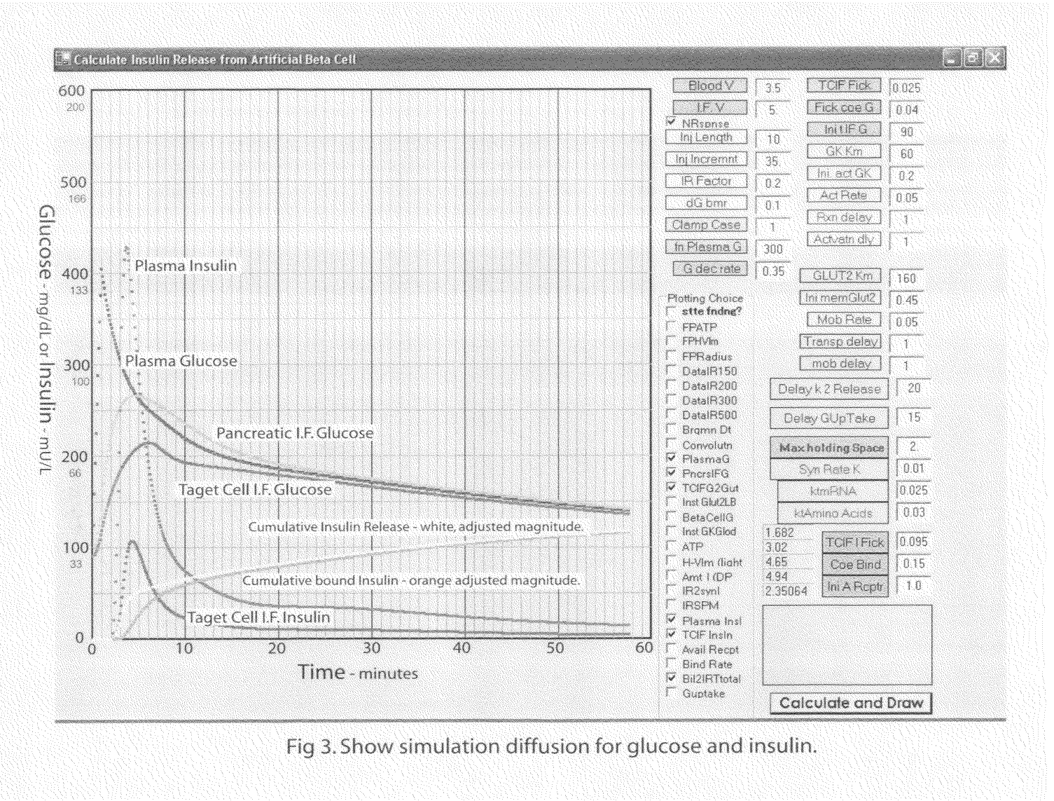

FIG. 3 shows effect of diffusion on glucose and insulin. Blood vessels, pancreatic interstitial and target cells interstitial fluids are the three involved compartments. Glucose level within three different compartments converging each other are shown on the top set of three curves in the figure. They are obtained via Fick's Law. This invention use glucose level around beta cell (pink curve) to establish the Gated Reservoir Model for insulin storage and secretion. The lower set of curves shows how insulin concentration in two compartments are approaching to each other. Insulin is bound to target cells and withdrawn from body fluids. The cumulative curves of insulin released and bound insulin prove that calculation is correct.

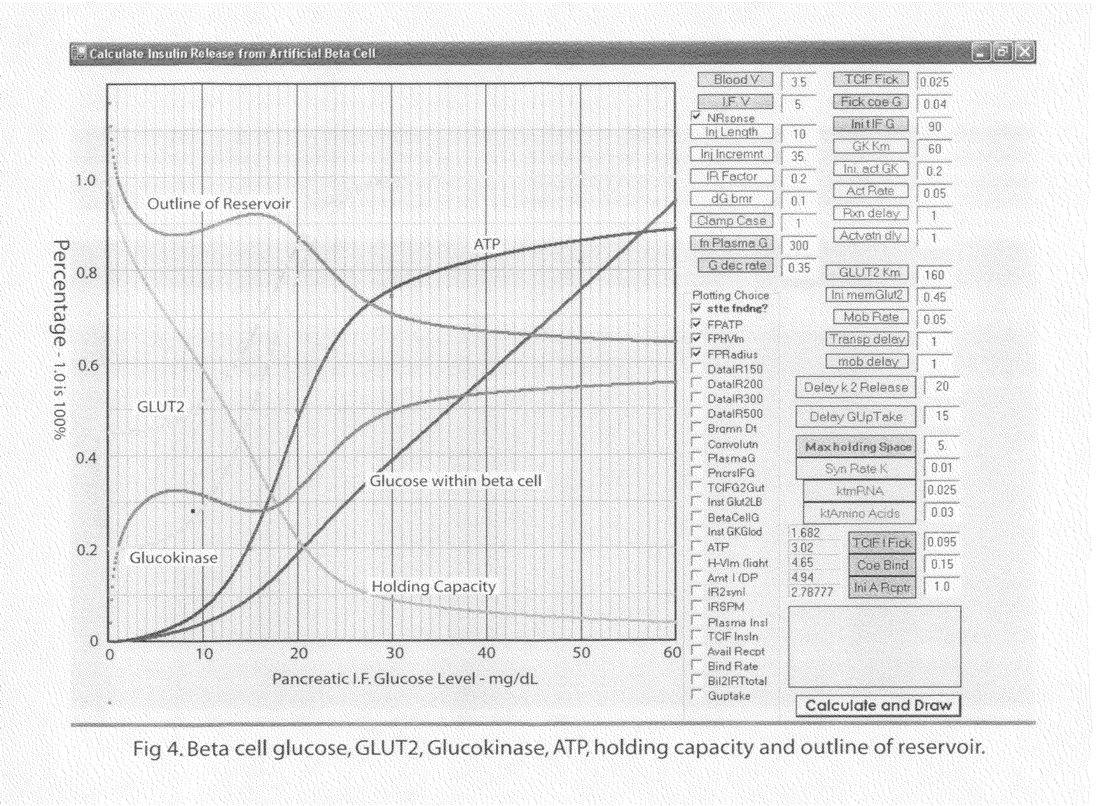

FIG. 4 shows the changes of GLUT2, GK, ATP and the outline of Gated Reservoir as function of glucose level of pancreatic fluids. Values are obtained from the Menten Equation in enzyme-kinetics. It is used recursively to obtained higher membrane GLUT2 concentration and same for activated glucokinase. ATP values is related to holding capacity directly. The outline of our reservoir (deep pink curve) is calculated and plotted for easier visualization.

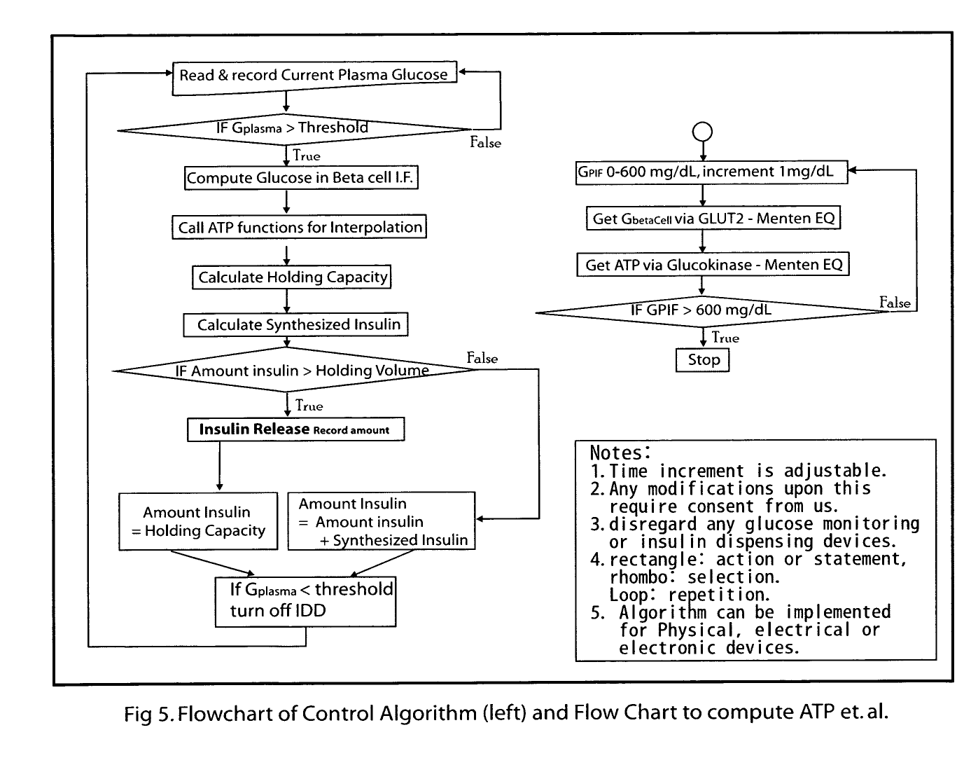

FIG. 5 shows a flow chart of the beta cell mimicking control algorithm based on GRM. It also shows a flow chart for obtaining ATP as a function of glucose level of pancreatic fluids.

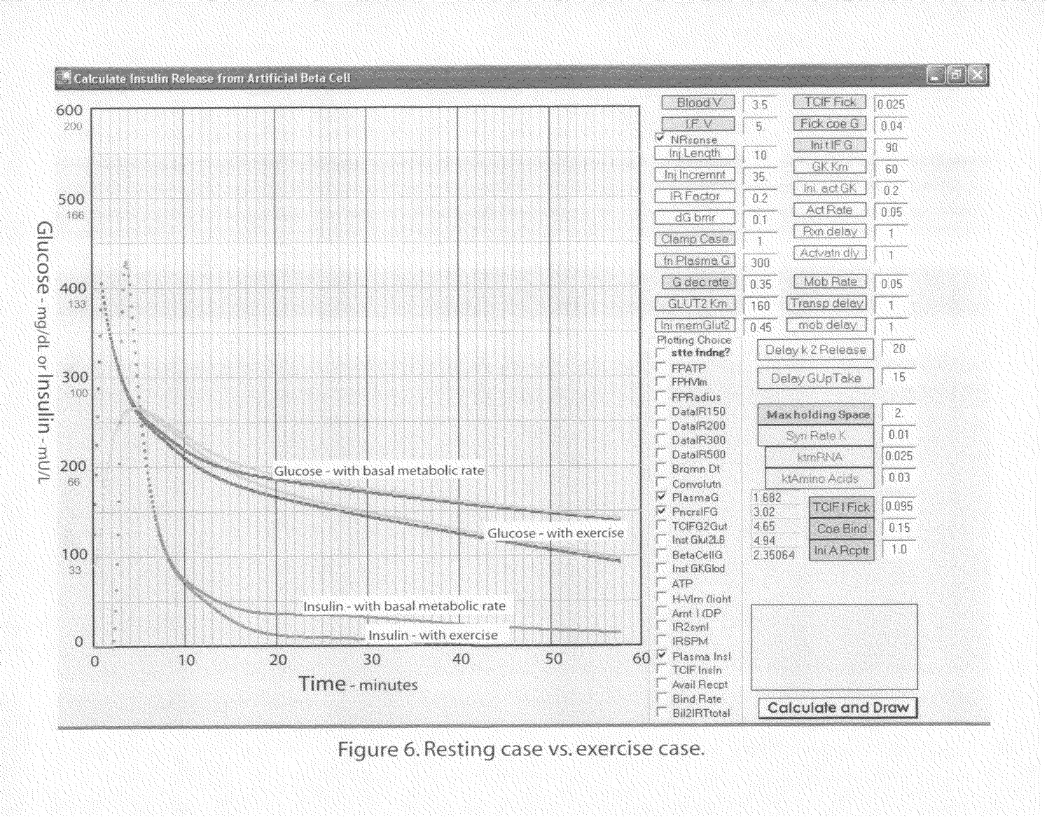

FIG. 6 shows a plasma glucose and plasma insulin variations for a resting case versus an exercise case. Extra glucose uptake via other pathway, exercise in this case, is set in the simulation. Our artificial pancreas control algorithm responses with less insulin release which is shown in plasma insulin variation. In a real life situation, a patient who exercises or moves more will receive less insulin from the IAPS. Their risk of having hypoglycemia will be minimum.

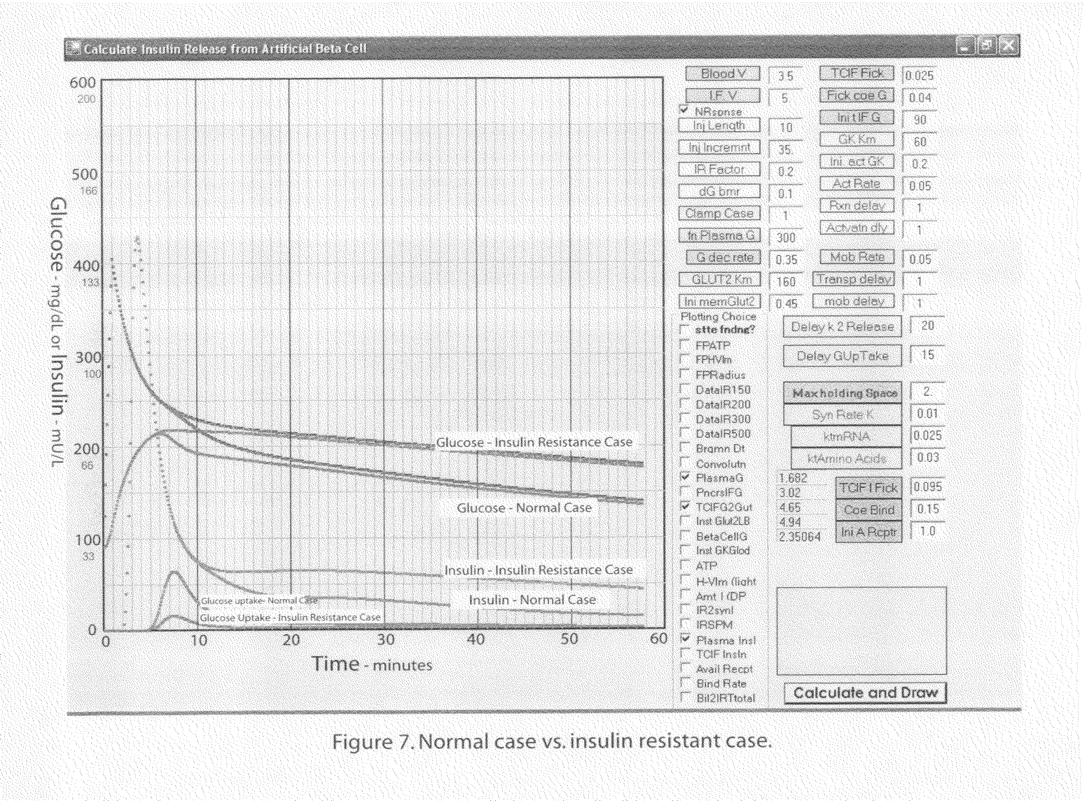

FIG. 7 shows the case of patient with Insulin Resistance (IR). We assign, four times more than a normal person, insulin resistance in this simulation. Glucose decreasing is slower for IR case. However, glucose in both cases increase to same level and decrease the same amount at around eighth minute. This decreasing of plasma glucose is due to diffusion, it is not due to the effect of insulin activated glucose uptake. More importantly, insulin released a lot more in IR case. IR can leads to hyperglycemia as well as hyperinsulinemia. Both conditions are great threats to the health of a person.

The control algorithm of this invention attempts to include as many bioprocesses as possible to simulate glucose metabolism of a normal person and to simulate the storage, synthesis and release of insulin from normal beta cell. An integrated artificial pancreas system with this control algorithm should allow patients of Type I diabetes to gain much better glycaemic control and to reduce their risk of further complications.

Inside human body, many organs are involved in glucose metabolism and therefore various bioprocesses have to work between and/or within them. FIG. 2 summarizes these 12 involved bioprocesses. Glucose-Insulin kinetics (the quantitative study of interactions and fluctuations between glucose and insulin) can only be understood and be applied through the understanding of all the above processes. However, our focus is on bioprocess 4 (insulin storage), 5 (insulin synthesis) 6, (insulin secretion) where a Gated Reservoir Model (GRM) is used to explain how insulin is secreted from beta cell. GRM, the core of this invention, also provide a quantitative real-time insulin release due to glucose variation. In FIG. 2, solid arrows indicate route of transported glucose. Outline arrows indicate route of transported insulin. Compartment stands for collective tissues, organs or interstitial spaces although they are not together as one unit physically. However, aggregate behavior of all those elements in the same compartment makes the study of glucose-insulin kinetics feasible.

In order to handle the changes of all the above bioprocesses, we take action-on-frozen-time approach. In other words, changes for a preset time interval (sampling time interval of CGS and IDD) of all 12 processes are computed bio-sequentially. It is called discrete time series in engineering terms. This simulation adopts six seconds as sampling rate for the convenience of a tenth of a minute. For each compartment, there is an input process and out process or processes. The first advantage of this approach is that only arithmetic is required for computation. No partial differential equations to be solved. It may not be robust mathematically yet simple enough to be implemented. Actual code is typically written using MicroSoft C++.net so that a simulation can run on most PCs. The algorithm would typically be independent of the hardware.

Bioprocess 1—Glucose Intake. Glucose enter blood streams by either absorption from intestine for a normal person or by intravenous injection in the case of a hospitalized patient. Since glucose sensors or glucose monitors measure plasma glucose directly, one can ignore the bioprocess of digestion and absorption. However, with glucose sensors, the IAPS can send a signal to a patient to stop food intake whenever blood glucose is high. For example, it exceeds 300 mg/dL (miligram per deci liter).

Bioprocess 2—Glucose Diffusion from Blood to Pancreatic Interstitial Fluids. Glucose exchanges between interstitial fluids around beta cells and surrounding capillaries through diffusion. The direction glucose move is determined by chemical gradient, from high concentration to low concentration. The amount transported is determined by Fick's law. Fick's law states that the net amount of solute (glucose in this case) transported per unit time across membrane is proportional to concentration difference.

Δ[G_{IF}]=k×[Gₚ]−[G_{IF}]

where Δ[G_{IF}] denotes glucose concentration change per unit time, k is Fick's coefficient, [Gₚ] is plasma glucose concentration and [G_{IF}] is glucose concentration of pancreatic interstitial fluids. Conservation of mass has to be incorporated into calculation. However, since the volume of pancreatic interstitial fluids is a small fraction of plasma volume, [G_{IF}] changes is set to be the fractional changes of the chemical gradient. The importance of getting glucose level of pancreatic interstitial fluids is due to the fact that beta cells response to their surrounding glucose level not plasma glucose level which is further away. Although IAPS is measuring plasma glucose, glucose level of pancreatic interstitial fluids can be found through the above formula.

Bioprocess 3—Glucose Transport. Glucose then enters beta cells via glucose transporter GLUT2. This process belongs to the category of enzymes-kinetics where Menten Equation explains quantitatively the reaction. Menten Equation states

ΔG=[GLUT2]×[S]/([S]+Kₘ)

where ΔG denotes amount of glucose transported into beta cells per unit time, [S] is substrate concentration and in this case is glucose concentration of pancreatic interstitial fluids, [GLUT2] denotes the amount of mobilized GLUT2−GLUT2 has to be on cell membrane in order to take glucose from pancreatic interstitial fluids into beta cells, Kₘ is Menten constant. Menten constant gives rise to the range of glucose concentration where glucose is transported most efficiently. The K value is set 160 mg/dL for this algorithm. In addition, the amount of membrane GLUT2 changes as more ATP (adenosine triphosphate—the molecular energy carrier) within beta cells increases. GLUT2 within cytosol get mobilized to cell membrane and becomes glucose taking GLUT2. A recursive Menten Equation has to be adopted here. Another concept of limited total GLUT2 has to be applied in order to have correct amount of membrane GLUT2.

Total GLUT2=Cytosol GLUT2+Membrane GLUT2 at any time.

Therefore, instead of being a constant as in the normal Menten Equation, [GLUT2] is increased from previous state in this recursive Menten Equation until almost all GLUT2 within cytosol are mobilized to cell membrane. A percentage membrane GLUT2 function of surrounding glucose concentration can be obtained to determine glucose concentration within beta cell.

The endocrine part of a pancreas consists of alpha cells, beta cells and delta cells. Beta cells, approximately 1 trillion of them, are responsible for insulin storage, synthesis and release. There are approximately ten thousands granules within each beta cell. Each granule packs 300 thousands insulin molecules within it. Five percentage of all the granules of human pancreas are readily releasable. It is the behavior of this insulin storage pool we want to decipher using Gated Reservoir Model. Apparently, beta cell also releases insulin from other pools to perform glucose metabolism. However, simulation of multi-reservoirs will be our next task.

Bioprocess 4—ATP Generation and Insulin Storage. Cells perform their various functions by pumping ions in and out against concentration gradient across cell membrane. Energy is required for those works. Glucose break down within beta cells cytosol then enters mitochondria (the molecular manufacturer of energy), generates chemical energy in a form of ATPs at the end. For ATPs generated within a beta cell, two percentage of them are used to regulate the ATP control potassium channel. Oscillating movement of potassium ions, sodium ions and calcium ions enable granules for exocytosis (granules got push out of cell). When more ATPs are provided, capacity of granules exocytosis will become higher.

The most critical step in generating ATPs is via glucokinase (an enzymes to assist ATP generation within mitochondria). This reaction belongs to the same enzymes kinetics where recursive Menten equation is adopted again. The same concept of limited glucokinase has to be applied too. In summary,

Membrane [GLUT2]+[G_{IF}]→[GbetaCell],

[GbetaCell]+[Glucokinase]→ATP,

Cytosol [GLUT2]+ATP→Membrane [GLUT2],

Inactive [Glucokinase]+ATP→Activated [Glucokinase].

Membrane [GLUT2] and activated [Glucokinase] are updated for every increment of [G_{IF}]. The result of this bioprocess calculation is a percentage ATP level as a function of surrounding glucose concentration. If we assign a total holding capacity to the reservoir (all granules within readily releasable pool of a pancreas) and assume expelling capacity is proportional to amount of ATP, we have

Holding Capacity=Total Holding Capacity×(1.−Percentage ATP).

Holding capacity has the same unit for any given insulin amount. The insulin unit is denoted as UNIT which is equivalent to 6 micrograms of insulin. FIG. 3 shows GLUT2, Glucokinase, ATP, Holding Capacity as function of glucose level of pancreatic interstitial fluids. If capacity volume is transformed into a three dimensional reservoir, the revolving radius outline (a vase shape) of the cone reservoir is shown in the same figure.

Part of the FIG. 5 shows a flow chart of the algorithm to obtain ATP function.

Bioprocess 5—Insulin Synthesis. Beta cells are working constantly to synthesize insulin. After synthesis, mature and active insulin are packed into granules and join to those pools that have been consumed. However, the rate of insulin synthesis is not high enough to provide post-prandial (after meal) glucose metabolism. A control algorithm based on a single reservoir (current version) can perform glucose uptake by setting a high synthesis rate. We use the following formula to simulate a high rate insulin synthesis.

I_{syn}=K_{syn}×(1.−exp(−αT))

Where I_{syn} is insulin synthesized per unit time, K_{syn} is amount factor, a reflect the rate of insulin synthesis. T denotes time after beta cell has available ATPs for synthesis.

Bioprocess 6—Gated Reservoir Model for Insulin Secretion. Since the variation of plasma glucose is not sufficient to determine insulin release from a beta cell, we adopt GRM to simulate the simplified beta cell response to glucose influx and to quantify its resultant insulin secretion. For a water reservoir such as one for an artificial lake, reservoir holding volume is determined by how high its gate is raised. Water spills out of reservoir only when water level is higher than the gate. In this model, holding capacity of beta cells corresponds to reservoir holding volume. Water amount in the reservoir corresponds to insulin amount within beta cells. A good analogy is that insulin can only be secreted into surrounding interstitial fluids when insulin amount is greater than a beta cell's holding capacity at that moment. Insulin secretion is a result of two relative quantities. In other words, lower holding capacity (due to higher glucose level) does not imply higher insulin release. In order to have insulin release, beta cell must have enough amount of insulin within that reservoir. Insulin level at any time within a beta cell is determined by the storage of insulin at that moment and amount of insulin synthesized during that moment.

I_{released}=Iₐₘₒᵤₙₜ−Holding Capacity,

Iₐₘₒᵤₙₜ=Iₚₐₘₒᵤₙₜ−I_{secreted}+I_{synthesized},

Where I_{released} is insulin got secreted to blood vessels per unit time. Iₐₘₒᵤₙₜ denotes insulin amount within beta cell at that moment. Iₚₐₘₒᵤₙₜ: insulin amount of previous moment, I_{secreted}: insulin secreted from previous moment, I_{synthesized}: insulin synthesized from previous moment. Holding Capacity reflects present moment ATPs status.

Therefore, insulin can be released at low glucose level and no insulin release at high glucose levels are all possible. The shape of reservoir in this model will determine the size and shape of the bolus phase (the first sharp and large insulin release). It also determine behaviors of beta cell at different glucose level.

Bioprocess 7—Insulin Diffusion to Target Cell Interstitial Fluids. Insulin secreted from beta cells enter pancreatic interstitial fluids. It traverses in the blood stream, diffuses out and distributes to tissues all over human body. Skeletal muscle cells, fat cells are all target cells of the insulin messenger. Insulin diffuses to interstitial fluids around all these target cells according to the same Fick's law. Since the volume of whole body interstitial fluids is in the same order of magnitude with the volume of blood, conservation of mass is important in getting the correct concentration change in both compartments.

Δ[Iₚ]×Vₚ=Δ[I_{IF}]×V_{IF},

where Δ[Iₚ] is insulin concentration change (becomes lower) within plasma, Vₚ is volume of blood, Δ[I_{IF}] is insulin concentration change (becomes higher) within interstitial fluids of whole human body and V_{IF} is volume of interstitial fluids of entire human body. Insulin concentration within target cells interstitial fluids can be evaluated with knowledge of insulin release. The information will be useful in finding the binding rate of insulin and in estimating glucose uptake.

Bioprocess 8—Glucose Diffusion to Target Cell Interstitial Fluids. Governed by the same Fick's law as in previous process, glucose diffuses to whole-body interstitial fluids. We also have

Δ[Gₚ]×V_{P}=Δ[G_{IF}]×V_{IF},

where Δ[Gₚ] is glucose concentration change within blood and A[G_{IF}] is glucose concentration change (becomes higher) within interstitial fluids of whole human body.

Bioprocess 9—Insulin Binding on Target Cells. Insulin has to bind on the receptors of a target cell in order to instruct cell to start glucose uptake. The amount of insulin bound to the cell's receptor is proportional to its concentration within interstitial fluids at that moment and is proportional to the amount of available receptors. We have binding rate as

I_{binded}=K_{b}×[I_{IF}]×[Receptors],

where I_{binded} is amount of bound insulin per unit time, [Receptors] is percentage receptors concentration and K_{b} is binding rate constant. We made an assumption in this computation that once receptors are occupied, it can not bind with other insulin during a post-prandial period.

Bioprocess 10—Glucose Uptake. Although this process as well as process 7 and 9 are not necessary in our IAPS, simulation of glucose uptake can greatly facilitate the search of a perfect IAPS. Glucose uptake per unit time is actually convolution (digital signal processing term) result of insulin binding rate and glucose uptake time history per insulin. And,

G_{UT}=I_{bindingRate}*G_{uptake/insulin}.

The convolution is just summing up the effect of current bound insulin and all previously bound insulin which are still uptaking glucose. Therefore time at t,

G_{UTt}=ΣI_{bindingRate(t-}τ)×G_{uptake/insulin} τ, τ is from 0 to the length of G_{uptake/insulin}.

With glucose uptake simulation, we are able to simulate an almost complete glucose metabolism of human body.

Bioprocess 11—Basal Metabolism. Neurons, liver cells and beta cells require glucose for metabolism at all times. Even if a person is resting, his or her body still consumes glucose for internal organ functions. We approximate this process by assigning a constant rate of basal metabolism for simplicity.

Bioprocess 12—Other Glucose Uptake pathway. There are other pathway to facilitate glucose uptake—exercise for example. We approximate this process in a similar way by assigning a constant rate of glucose uptake for certain time period. The effect of exercise on insulin release can be shown with our complete simulation.

Although there are 12 bioprocesses involved in glucose metabolism, only six processes are needed for simulation in constructing the control algorithm of an artificial pancreas. These are process 2 through 6. Technically, process 2 is used to obtained glucose level of pancreatic interstitial fluids form plasma glucose reading via Fick's law. An ATP function of glucose level of pancreatic interstitial fluids is established from simulation of processes 3 and 4. This function with interval of 1 mg/dL, has to be computed and preset before IAPS starts working. When a glucose level of pancreatic interstitial fluids is computed, two values of above function, which are values of two adjacent glucose level of pancreatic interstitial fluids, are called for interpolation to find current ATP value. Algorithm then computes current holding volume. At the same time, current amount of insulin is evaluated from previous time insulin storage and insulin synthesis. The difference between amount of insulin and holding volume determines amount of insulin release. If amount of insulin is larger than holding volume, that amount of insulin is released. If smaller, there will be no release and that amount of synthesized insulin is added to amount of insulin in the reservoir. The algorithm then go on to input next reading of plasma glucose level. The procedure will continue until a goal plasma glucose level is reached. Current flowchart for the control algorithm of our IAPS is shown in FIG. 5.

IAPS should perform continuous glucose monitoring. However, IDD can be set to start with a threshold plasma glucose reading, exceeding 100 mg/dL for example. IDD should be turned off with a threshold reading too for example, below 100 mg/dL.

Exercise provides another pathway for glucose uptake. Plasma glucose and plasma insulin variations, FIG. 6, obtained from such simulation is tested against a resting case (with only small basal metabolic rate). Our artificial pancreas control algorithm responses with less insulin release which is shown in the plasma insulin variation curve. In a real life situation, patient who exercises or moves more will receive less insulin form our IAPS. There is much less risk to have hypoglycemia.

Insulin Resistance (IR) case is very common among Type II diabetic patients. We assign, four time more than a normal person, insulin resistance to the simulation. FIG. 7 shows plasma glucose and plasma insulin concentration variations due to IR. Glucose decrease is slower for an IR case. However, glucose in both cases increases to the same level and decrease the same amount at around eighth minute. This decreasing of plasma glucose is due to diffusion, it is not due to the effect of insulin-activated glucose uptake. A insulin resistance index can be defined as the ratio cumulative insulin release over cumulative plasma glucose decrease. Of course, diffusion effect must be excluded from estimating amount of glucose decrease. More importantly, insulin released a lot more in IR case. IR can leads to hyperglycemia as well as hyperinsulinemia. Both conditions are great threats to the health of a person. Fortunately, our IAPS can inform patient of insulin resistance on a real-time basis. In other words, it can tell how good your administered insulin is working for your body instantly. Those might be cases of ineffective insulin, body's insulin resistance or malfunction of IDD. Under such situation, IAPS should stop insulin release and notify support center or call patient's doctor. Normal beta cells do not sense whether their companion target cells have insulin resistance or not. In this aspect, our IAPS can be better than real beta cells.

Both glucose monitoring and insulin dispensing technology are widely available. Advanced and stable glucose monitoring devices with little invasive magnitude and higher sampling rates are available. However, the minimum sampling rate (times per minute) requirement of a glucose sensor for an artificial beta cell to function properly has to be at least a few times in one minute. Overall, glucose sensor has to catch the rapid increase of plasma glucose right after food intake to have proper first bolus release.

This control algorithm is programmed on the CPU or electronics of the integrated artificial pancreas system, which can take a number of forms of physical embodiments but generally attached to the control unit that looks like a pack or box strapped to a person. This algorithm does not require Karlman filter for error estimation on glucose reading. Although, the successfulness of any artificial pancreas relies on an accurate reading of blood glucose.

Due to patient's individuality, quality of manufactured insulin and physiological improvements, supports to doctors and patients for analyzing glucose-insulin kinetics for particular patients should be provided. This support can be established via the device's recording and communication capabilities and via a data analysis center where glucose variation and the amount of insulin dispensed can be collected and analyzed. Recorded data can be wirelessly transmitted to a central monitoring station, or a patient support center. With communication capability, IAPS can also be updated with new version of control algorithm or accept a different set of parameters. In addition, in case of an abnormal situation, the patient support center can notify doctor and patient.

1. A method of controlling an artificial pancreas comprising the steps of:
a. presetting total holding capacity;
b. approximating beta cell insulin reservoir current holding volume from reading a glucose level; wherein insulin reservoir holding capacity reflects present moment ATP status;
c. calculating a previous moment insulin amount, calculating a previous moment insulin secreted amount, calculating a previous moment insulin synthesized amount;
d. calculating a suggested insulin dose which equals the previous moment insulin amount−previous moment insulin secreted amount+previous moment insulin synthesized amount;
e. outputting the suggested insulin dose;
f. repeating steps b-f.

2. The method of claim 1, further comprising the step of: collecting data for analysis via a computer network to adjust artificial pancreas parameters.

3. The method of claim 1, further comprising the step of: notifying patient upon abnormal events.

4. The method of claim 1, further comprising the step of: aggregating data at a data analysis center.

5. The method of claim 1, further comprising the step of: using patient data history to determine insulin sensitivity.

6. The method of claim 1, further comprising the step of: recording and communication with devices in IAPS.

7. The method of claim 1, further comprising the step of: sending both glucose and insulin data for analysis.

8. The method of claim 1, further comprising the step of: repeating step a.

9. The method of claim 1, further comprising the step of: using glucose level of pancreatic interstitial fluids and subsequent computation to calculate suggested insulin.

10. The method of claim 1, further comprising the step of: assigning a reservoir shape obtained from enzyme kinetics with ATP feedback to geometrically model the rate of change of beta cell insulin reservoir current holding volume.

11. A method of controlling an artificial pancreas comprising the steps of:
a. reading and recording a current plasma glucose level;
b. computing glucose in beta cell if a current plasma glucose level is greater than a threshold value, and continuing the step of reading and recording a current plasma glucose level if a current plasma glucose level is less than a threshold value;
c. calling ATP functions for interpolation;
d. calculating a holding capacity;
e. calculating a synthesized insulin amount;
f. comparing amount of insulin with the holding capacity;
g. releasing insulin if the amount of insulin is greater than the holding capacity, and not releasing insulin if the amount of insulin is less than the holding capacity;
h. repeating steps a-g.

12. The method of claim 11, further comprising the step of: collecting data for analysis via a computer network to adjust artificial pancreas parameters.

13. The method of claim 11, further comprising the step of: notifying patient upon abnormal events.

14. The method of claim 11, further comprising the step of: aggregating data at a data analysis center.

15. The method of claim 11, further comprising the step of: using patient data history to determine insulin sensitivity.

16. The method of claim 11, further comprising the step of: recording and communication with devices in IAPS.

17. The method of claim 11, further comprising the step of: sending both glucose and insulin data for analysis.

18. The method of claim 11, further comprising the step of: assigning a reservoir shape obtained from enzyme kinetics with ATP feedback to geometrically model the rate of change of beta cell insulin reservoir current holding volume.
